SEQUENCE LISTING (17 sequences)

SEQ ID NO: 1 - protein, 248 aa, Artificial sequence
  Synthetic
MWLCPLALNLILMAASGAACEVKDVCVGSPGIPGTPGSHGLPGRDGRDGVKGDPGPPGPMGPPGETPCPPGNNGLPGAPGVPGERGEKGEAGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 2 - protein, 10 aa, Artificial sequence
  Synthetic
KEQCVEMYTD

SEQ ID NO: 3 - protein, 12 aa, Artificial sequence
  Synthetic
WGKEQCVEMYTD

SEQ ID NO: 4 - protein, 10 aa, Artificial sequence
  Synthetic
KEQCVEMYTD

SEQ ID NO: 5 - protein, 10 aa, Artificial sequence
  Synthetic
KEQCVEMYTD

SEQ ID NO: 6 - protein, 10 aa, Artificial sequence
  Synthetic
KEQCVEMYTD

SEQ ID NO: 7 - protein, 10 aa, Artificial sequence
  Synthetic
  X (7)..(7): X is Nle
KEQCVEXYTD

SEQ ID NO: 8 - protein, 10 aa, Artificial sequence
  Synthetic
KEQSVEMYTD

SEQ ID NO: 9 - protein, 10 aa, Artificial sequence
  Synthetic
KEQAVEMYTD

SEQ ID NO: 10 - protein, 23 aa, Artificial sequence
  Synthetic
SDGTPVNYTNWYRGEPAGRGKEQ

SEQ ID NO: 11 - protein, 20 aa, Artificial sequence
  Synthetic
GDFRYSDGTPVNYTNWYRGE

SEQ ID NO: 12 - protein, 12 aa, Artificial sequence
  Synthetic
  X (9)..(9): X is Nle
WGKEQAVEXYTD

SEQ ID NO: 13 - protein, 12 aa, Artificial sequence
  Synthetic
  X (9)..(9): X is Nle
WGKEQCVEXYTD

SEQ ID NO: 14 - protein, 12 aa, Artificial sequence
  Synthetic
  X (9)..(9): X is Nle
RGKEQCVEXYTD

SEQ ID NO: 15 - protein, 11 aa, Artificial sequence
  Synthetic
  misc_feature (8)..(8): Xaa can be any naturally occurring amino acid
GKEQCVEXYTD

SEQ ID NO: 16 - protein, 10 aa, Artificial sequence
  Synthetic
KEQCVEMYTD

SEQ ID NO: 17 - protein, 20 aa, Artificial sequence
  Synthetic
PAGRGKEQCVEMYTDGQWND

(19) United States
(12) Patent Application Publication
(10) Pub. No.: US 2023/0135877 A1
(43) Pub. Date: May 4, 2023

(54) COMPOSITIONS AND METHODS FOR TREATING AND PREVENTING LUNG DISEASE

(71) Applicant: Arizona Board of Regents on Behalf of the University of Arizona, Tucson, AZ (US)

(72) Inventors: Julie Ledford, Tucson, AZ (US); Monica Kraft, Tucson, AZ (US); Josef Vagner, Tucson, AZ (US); Scott Boitano, Tucson, AZ (US)

(21) Appl. No.: 17/917,790

(22) PCT Filed: Apr. 8, 2021
(86) PCT No.: PCT/US2021/026411

§ 371 Date: Oct. 7, 2022

Related U.S. Application Data
(60) Provisional application No. 63/006,831, filed on Apr. 8, 2020.

(51) Int. Cl. C07K 14/785; A61P 11/06

(52) U.S. Cl. CPC C07K 14/785; A61P 11/06

(57) ABSTRACT

Provided herein are compositions and methods for treating and preventing lung disease. In particular, provided herein are surfactant protein-A (SP-A) peptide analogies (e.g., SP-A peptidomimetics) and uses thereof in the treatment and prevention of lung disease (e.g., asthma or COPD).

### CROSS REFERENCE TO RELATED APPLICATION

This application claims the benefit of U.S. Prov. Appl. 63/006,831 filed Apr. 8, 2020, the entire contents of which are incorporated herein by reference.

### FIELD OF THE INVENTION

Provided herein are compositions and methods for treating and preventing lung disease. In particular, provided herein are surfactant protein-A (SP-A) peptide analogues (e.g., SP-A peptidomimetics) and uses thereof in the treatment and prevention of lung disease (e.g., asthma or COPD).

### BACKGROUND OF THE INVENTION

Asthma is the most common respiratory disease in both children and adults, and presents as a syndrome of non-specific airways hyperresponsiveness, inflammation and intermittent respiratory symptoms affecting 10% of the population (Bousquet et al. Bull World Health Organ. 2005; 83 (7):548-54. PubMed PMID: 16175830; Mannino et al. Surveillance for asthma--United States, 1980-1999. MMWR Surveill Summ. 2002; 51(1):1-13. PubMed PMID: 12420904). It is triggered by infection, environmental allergens or other stimuli (Bousquet et al. Bull World Health Organ. 2005; 83(7):548-54. PubMed PMID: 16175830; Mannino et al. Surveillance for asthma--United States, 1980-1999. MMWR Surveill Summ. 2002; 51(1):1-13. PubMed PMID: 12420904).

Asthma remains poorly understood and difficult to manage in many cases due to the heterogeneity of the disease. A significant cause of morbidity and mortality in asthma is the acute exacerbation, which can lead to airway injury, remodeling, lung function decline and death. (Halwani R, et al. Curr Opin Pharmacol. 2010; 10(3):236-45. PubMed PMID: 20591736; Firszt R, Kraft M. Pharmacotherapy of severe asthma. Curr Opin Pharmacol. 2010; 10(3):266-71. PubMed PMID: 20462794). Most exacerbations are caused by respiratory infection such as rhinovirus or Mycoplasma pneumoniae. The response to infection is complex, involving both the innate and adaptive immune system (Kim HY et al. The many paths to asthma: phenotype shaped by innate and adaptive immunity. Nat Immunol. 2010; 11(7):577-84. PubMed PMID: 20562844). Exacerbations in more severe asthmatics are of particular concern, as hospitalizations for acute exacerbations account for one-third of the $14.7 billion dollars spent annually on asthma-related health care in the US. In addition, exacerbations in this population are associated with accelerated lung function decline (Peat JK, Woolcock AJ, Cullen K. Rate of decline of lung function in subjects with asthma. Eur J Respir Dis. 1987; 70(3):171-9. PubMed PMID: 3569449; Peat JK, Woolcock AJ, Cullen K. Rate of decline of lung function in subjects with asthma. Eur J Respir Dis. 1987; 70(3):171-9. PubMed PMID: 3569449). As reduced lung function is a risk factor for severe exacerbation (Osborne ML, Pedula KL, O′Hollaren M, Ettinger KM, Stibolt T, Buist AS, et al. Assessing future need for acute care in adult asthmatics: the Profile of Asthma Risk Study: a prospective health maintenance organization-based study. Chest. 2007; 132(4):1151-61. PubMed PMID: 17573515; Dougherty R H, Fahy J V. Acute exacerbations of asthma: epidemiology, biology and the exacerbation-prone phenotype. Clin Exp Allergy. 2009; 39(2):193-202. PubMed PMID: 19187331), this vicious cycle can promote an exacerbation-prone phenotype of asthma. Thus, an understanding of the mechanisms driving asthma exacerbations has been a critical barrier to progress in the understanding of asthma pathobiology.

Intact immune system and host defense functions are critical to preventing exacerbations of asthma. Surfactant is a lipoprotein complex that reduces surface tension at the air-liquid interface of the lung and participates in host defense (Han S, Mallampalli RK. The role of surfactant in lung disease and host defense against pulmonary infections. Annals of the American Thoracic Society. 2015; 12(5):765-74. Epub 2015/03/06. doi: 10.1513/AnnalsATS.201411-507FR. PubMed PMID: 25742123). The pulmonary surfactant system of the lung is an extracellular lipid and protein complex, present at the air/tissue interface, which regulates both the biophysical properties of the alveolar compartment, and the innate immune system of the organ. It has been shown that surfactant protein A (SP-A) promotes key cellular functions that can attenuate the severity of the disease and the exacerbation, which includes enhancing apoptosis of eosinophils, a critical cell in asthma pathobiology, reduce mucin production by airway epithelial cells in the setting of interleukin (IL)-13 exposure, a cytokine essential to the allergic asthma phenotype and reduces IL-6 production, another cytokine important in type 2 or allergic inflammation.

Airway inflammation is a hallmark feature of asthma. Eosinophils are prominent in individuals with a type 2 inflammatory asthma phenotype, and accrue in large numbers in the circulation, sputum, and airway mucosa (see, e.g., Wenzel, S. E., Nature medicine, 2012. 18(5): p. 716-25). Eosinophil accumulation and prolonged viability in the airways is strongly correlated with greater asthma severity (see, e.g., Green, R. H., et al., Lancet, 2002. 360(9347): p. 1715-21; Duncan, C. J., et al., The European respiratory journal, 2003. 22(3): p. 484-90; Gibson, P. G., et al., Thorax, 2003. 58(2): p. 116-21; Leitch, A. E., et al., Mucosal immunology, 2008. 1(5): p. 350-63) and their presence is driven by the type 2 cytokines interleukin (IL)-4, 5 and 13. Recent studies have shown that within the group of severe asthmatics, approximately 50% have eosinophils present in their lung tissues (see, e.g., Wenzel, S. E., et al., American journal of respiratory and critical care medicine, 1999. 160(3): p. 1001-8; Wenzel, S. E., Asthma phenotypes: the evolution from clinical to molecular approaches. Nature medicine, 2012. 18(5): p. 716-25). Moreover, treatment strategies targeted at reducing eosinophils have been shown to reduce asthma admission rates and exacerbations (see, e.g., Green, R. H., et al., Lancet, 2002. 360(9347): p. 1715-21; Jayaram, L., et al., The European respiratory journal, 2006. 27(3): p. 483-94). Clearance and rapid removal of apoptotic cells is an important process leading to the resolution of inflammation and mitigation of asthma symptoms. Inefficient apoptotic cell clearance results in secondary necrosis or cytolysis, the release of cellular contents that can damage tissue, and prolong inflammation and duration of asthma symptoms. Additionally, asthma severity is strongly correlated with prolonged eosinophil viability (see, e.g., Duncan, C. J., et al., The European respiratory journal, 2003. 22(3): p. 484-90; Fitzpatrick, A. M., et al., The Journal of allergy and clinical immunology, 2008. 121(6): p. 1372-8, 1378 el-3; Leitch, A. E., et al., Relevance of granulocyte apoptosis to resolution of inflammation at the respiratory mucosa. Mucosal immunology, 2008. 1(5): p. 350-63). Interestingly, inhaled beta-2 agonists, which are the mainstay of asthma treatment worldwide, have been shown to prolong eosinophil survival ((see, e.g., Nielson, C. P. and N. E. Hadjokas, American journal of respiratory and critical care medicine, 1998. 157(1): p. 184-91) and may actually exacerbate asthma or at least contribute to the variable response seen with beta-2 agonists (see, e.g., Choudhry, S., et al., Pharmacogenetics and genomics, 2010. 20(6): p. 351-8).

Additional treatments for asthma are needed.

The present invention addresses this need.

### SUMMARY OF THE INVENTION

Surfactant protein A (SP-A) is the most abundant protein component of the lipoprotein complex, pulmonary surfactant. In humans, full-length oligomeric SP-A is a product of SP A1 and SP-A2 genes. Although alveolar type II cells in the distal airway are the main producers of SP-A, it is synthesized independently of pulmonary surfactant in the conducting airways by club cells and submucosal glands (see, Auten, R. L., et al., 1990 Am J Respir Cell Mol Biol 3: 491-496; Goss, K. L., 1998 Am J Respir Cell Mol Biol 19: 613-621). In the nasal mucosa, SP-A can be detected in the cytoplasm of ciliated epithelial cells, serous acini and submucosal glands (see, Kim, J. K., et al., 2007 Am J Physiol Lung Cell Mol Physiol 292: L879-884; Wootten, C. T., et al., 2006 Arch Otolaryngol Head Neck Surg 132: 1001-1007; Woodworth, B. A., et al., 2006 Am J Rhinol 20: 461-465).

SP-A plays an important role in modulating type 2-associated allergen-induced inflammation. Mice deficient in SP-A have significantly increased type 2-associated cytokine levels, IgE levels and most notably, eosinophil levels compared to wild-type mice upon challenge with ovalbumin (OVA) (see, Pastva, A. M., et al., 2011 J Immunol 186: 2842-2849). Obese asthmatics, who have decreased levels of SP-A, have more severe tissue eosinophilia, and treatment with exogenous SP-A in mouse models of asthma has been shown to significantly reduce tissue eosinophilia (see, Lugogo, N., et al., 2017 J Allergy Clin Immunol.; Desai, D., et al., 2013 Am J Respir Crit Care Med 188: 657-663; van der Wiel, E., et al., 2014 Am J Respir Crit Care Med 189: 1281-1284). Further, SP-A isolated from asthmatics were unable to attenuate the production of airway epithelial IL-8 and Muc5ac in the context of Mycoplasma pneumoniae (Mp) infection, a bacteria highly associated with asthma exacerbations, compared to SP-A isolated from non-asthmatics (see, Wang, Y., et al., 2011 Am J Physiol Lung Cell Mol Physiol 301: L598-606).

It has been shown that the single nucleotide polymorphism that substitutes the glutamine (Q) for a lysine (K) at position 223 within SP-A2 results in altered eosinophil regulation in allergic airway inflammation (see, Dy, A. B. C., et al., 2019 J Immunol 203: 1122-1130) (G1n223Lys) (223Q/K within the SP-A wild type amino acid sequence shown at SEQ ID NO: 1). More specifically, SP-A2 that has this Q to K amino acid substitution, fails to promote eosinophil apoptosis, compared to SP-A2 that contains a Q at position 223. Additionally, it has been shown that the presence of a Q at this position is protective against respiratory insults (see, Lofgren, J., et al., 2002 J Infect Dis 185: 283-289; Marttila, R., et al., 2003 Ann Med 35: 344-352). Such findings highlight the relevance of this active region within SP-A for achieving normal airway function.

```
Human Wild Type amino acid sequence for SP-A
(SEQ ID NO: 1)
                10 20 30 40
MWLCPLALNL ILMAASGAAC EVKDVCVGSP GIPGTPGSHG
                50 60 70 80
LPGRDGRDGV KGDPGPPGPM GPPGETPCPP GNNGLPGAPG
                90 100 110 120
VPGERGEKGE AGERGPPGLP AHLDEELQAT LHDFRHQILQ
              130 140 150 160
TRGALSLQGS IMTVGEKVFS SNGQSITFDA IQEACARAGG
              170 180 190 200
RIAVPRNPEE NEAIASFVKK YNTYAYVGLT EGPSPGDFRY
              210 220 230 240
SDGTPVNYTN WYRGEPAGRG KEQCVEMYTD GQWNDRNCLY
SRLTICEF
```

Eosinophils are well-known end-stage effector cells and are major contributors to symptoms experienced in type 2 high asthma. Inhaled corticosteroid therapy, which aids in reducing eosinophil viability by inhibiting production of eosinophil-specific chemokines (see, Stellato, C., et al., 1999 J Immunol 163: 5624-5632) and cytokines that promote eosinophil survival (see, Schleimer, R. P., and B. S. Bochner. 1994. J Allergy Clin Immunol 94: 1202-1213), is a highly effective treatment strategy for asthma symptoms and exacerbations. Thus, it could be inferred that eosinophil apoptosis and their subsequent clearance is an important step in the resolution of type 2 associated airway inflammation.

By inhibiting eosinophil survival, corticosteroids may be considered an eosinophil normalization treatment strategy. This is in contrast to eosinophil depletion treatment strategies using biologics, such as Mepolizumab, Reslizumab (anti-IL 5 antibodies) and Benralizumab (anti-IL5Rα antibodies), the goal of which is to dramatically reduce circulating eosinophils and also their maturation in the bone marrow (see, Roufosse, F. 2018. Front Med (Lausanne) 5: 49). Despite inhaled corticosteroid therapy being a mainstay for the treatment of asthma, steroid resistance remains a challenge, along with the known side effects associated with this type of long-term therapy. Although biologics currently in clinical trials and in the market are steroid-sparing, long-term effects of eosinophil depletion are still unknown.

Preliminary data suggest that SP-A native peptides, 10 and 20 amino acids in length (10-mer and 20-mer), derived from the active site spanning position 223 can reduce airway hyperresponsiveness, airway mucus production and eosinophilia in a pre-clinical mouse model of asthma (FIG. 1).

Experiments conducted during the course of developing embodiments for the present invention developed small molecules with improved stability and bio-availability, derived from the SP-A active site, as therapeutics targeting eosinophil normalization. Indeed, such experiments screened the 10-mer and 20-mer SP-A peptides, along with a series of peptidomimetics derived from full-length SP-A for their direct apoptosis-promoting functions on eosinophils. Several potential peptidomimetics were identified that closely resemble full-length SP-A in reference to the cytotoxic effect of SP-A on eosinophils in vitro. Such results represent a proof-of-concept that small molecules derived from the active site of SP-A possess activity against eosinophils and paves the way for development of a new class of therapeutics for allergic airway inflammation.

Accordingly, provided herein are compositions and methods for treating and preventing lung disease. In particular, provided herein are SP-A peptide analogues (e.g., SP-A peptidomimetics) and uses thereof in the treatment and prevention of lung disease (e.g., asthma or COPD).

For example, in some embodiments, a composition comprising a peptide analogue comprising an amino acid sequence selected from, for example, Ac-KEQCVEMYTD-NH₂ (SEQ ID NO: 2), Ac-WGKEQCVEMYTD- NH₂ (SEQ ID NO: 3), (Ac-KEQCVEMYTD-NH₂)₂ (SEQ ID NO: 4), Ac-KEQCVEMYTD-acid (SEQ ID NO: 5), H-KEQCVEMYTD-acid (SEQ ID NO: 6), Ac-KEQCVE-Nle-YTD-NH₂ (SEQ ID NO: 7), Ac-KEQSVEMYTD-NH₂ (SEQ ID NO: 8), Ac-KEQAVEMYTD-NH₂ (SEQ ID NO: 9), Ac-SDGTPVNYTNWYRGEPAGRGKEQ-NH₂ (SEQ ID NO: 10), Ac-GDFRYSDGTPVNYTNWYRGE-NH₂ (SEQ ID NO: 11), Ac-WGKEQAVE-Nle-YTD-NH₂ (SEQ ID NO : 12), Ac-WGKEQCVE-Nle-YTD-NH₂ (SEQ ID NO: 13), Ac-RGKEQCVE-Nle-YTD-NH₂ (SEQ ID NO : 14), Ac-wGKEQCVE-Nle-YTD-NH₂ (SEQ ID NO: 15), or peptide analogues with at least 90% (e.g., 91%, 92%, 93%, 94%, 95%, 96%, 97%, 98%, 99%, or 100%) identity to the peptides is provided. Further embodiments provide a composition consisting essentially of a peptide analogue selected from, for example, Ac-KEQCVEMYTD-NH₂ (SEQ ID NO: 2), Ac-WGKEQCVEMYTD- NH₂ (SEQ ID NO: 3), (Ac-KEQCVEMYTD-NH₂)₂ (SEQ ID NO: 4), Ac-KEQCVEMYTD-acid (SEQ ID NO: 5), H-KEQCVEMYTD-acid (SEQ ID NO: 6), Ac-KEQCVE-Nle-YTD-NH₂ (SEQ ID NO: 7), Ac-KEQSVEMYTD-NH₂ (SEQ ID NO: 8), Ac-KEQAVEMYTD-NH₂ (SEQ ID NO: 9), Ac-SDGTPVNYTNWYRGEPAGRGKEQ-NH₂ (SEQ ID NO: 10), Ac-GDFRYSDGTPVNYTNWYRGE-NH₂ (SEQ ID NO: 11), Ac-WGKEQAVE-Nle-YTD-NH₂ (SEQ ID NO : 12), Ac-WGKEQCVE-Nle-YTD-NH₂ (SEQ ID NO: 13), Ac-RGKEQCVE-Nle-YTD-NH₂ (SEQ ID NO : 14), Ac-wGKEQCVE-Nle-YTD-NH₂ (SEQ ID NO: 15). In some embodiments, the composition is a pharmaceutical composition. In some embodiments, the composition comprises a pharmaceutically acceptable carrier. In some embodiments, the composition is formulated for pulmonary delivery.

Further embodiments provide a system, comprising: a) any one of the compositions described herein; and b) a device for pulmonary delivery of the composition. In some embodiments, the device is a metered dose inhaler.

Additional embodiments provide a method of enhancing SP-A activity in a cell, comprising: delivering any one of the compositions described herein to a cell. In some embodiments, the cell is a lung cell. In some embodiments, the cell is in vivo. In some embodiments, the composition reduces mucin production and/or reduces eosinophilia in the lung. In some embodiments, the cell is in a subject diagnosed with asthma. In some embodiments, the administering decreases or prevents symptoms or markers of asthma in the subject. In some embodiments, subject is obese or is not obese. In some embodiments, the peptide binds to a receptor selected from, for example FC (CD16/32), Sirp-alpha, TLR-2, or EGFR.

Still other embodiments provide a method of treating or preventing a lung disease (e.g., asthma or COPD) in a subject, comprising: administering any one of the compositions described herein to the subject.

Yet other embodiments provide the use of any one of the compositions described herein to enhance SP-A activity in a cell. Other embodiments provide the use of any one of the compositions described herein to treat or prevent lung disease (e.g., asthma or COPD) in a subject.

Additional embodiments are described herein.

### BRIEF DESCRIPTION OF THE DRAWINGS

FIG. 1A-C. Evaluation of SP-A-derived 10-mer native peptide in an in vivo mouse model of asthma. A) Schematic of HDM experimental allergen challenge. B) Newtonian resistance

(Rn) during methacholine challenge of wild-type mice 6 days after terminal HDM challenge. C) Total eosinophil counts in BAL (left panel) and mucin production (right panel) by PAS scoring. Unpaired t-test, *p<0.05, **p<0.01.

FIG. 2A-F. Evaluation of the cytotoxic effect of full-length SP-A and native peptides on eosinophils by RTCA. Normalized cell indices and calculated areas under the curve for each dose are shown for SP-A (A-B), 20-mer peptide (C-D) and 10-mer peptide (E-F).

FIG. 3A-B. Evaluation of the cytotoxic effect of candidate peptidomimetics on eosinophils by RTCA using mass concentration. Normalized cell indices (A) and calculated areas under the curve (B) for each dose are shown for 856, 867, 868, 870, 871, 882, 883 and 884.

FIG. 4A-B. Evaluation of the cytotoxic effect of candidate peptidomimetics on eosinophils by RTCA using molar concentration. Normalized cell indices (A) and calculated areas under the curve (B) for each dose are shown for 888, 889, 891, 892, 893 and 894.

### DEFINITIONS

The terms “polypeptide” and “protein” are used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition. The terms also include post-translational modifications of the polypeptide, including, for example, glycosylation, sialylation, acetylation, and phosphorylation. Furthermore, a “polypeptide” herein also refers to a modified protein such as single or multiple amino acid residue deletions, additions, and substitutions to the native sequence, as long as the protein maintains a desired activity. For example, a serine residue may be substituted to eliminate a single reactive cysteine or to remove disulfide bonding or a conservative amino acid substitution may be made to eliminate a cleavage site. These modifications may be deliberate, as through site-directed mutagenesis, or may be accidental, such as through mutations of hosts, which produce the proteins or errors due to polymerase chain reaction (PCR) amplification.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

“Wildtype” refers to a non-mutated version of a gene, allele, genotype, polypeptide, or phenotype, or a fragment of any of these. It may occur in nature or be produced recombinantly.

A “variant” is a nucleic acid molecule or polypeptide that differs from a referent nucleic acid molecule or polypeptide by single or multiple amino acid substitutions, deletions, and/or additions and substantially retains at least one biological activity of the referent nucleic acid molecule or polypeptide.

The terms “peptide mimetic” or “peptidomimetic” refer to a peptide-like molecule that emulates a sequence derived from a protein or peptide. A peptide mimetic or peptidomimetic may contain amino acids and/or non-amino acid components. Examples of peptidomimitecs include chemically modified peptides, peptoids (side chains are appended to the nitrogen atom of the peptide backbone, rather than to the α-carbons), (β-peptides (amino group bonded to the β carbon rather than the a carbon), etc.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

“Subject,” “individual,” “host,” “animal,” and “patient” are used interchangeably herein to refer to mammals, including, but not limited to, rodents, simians, humans, felines, canines, equines, bovines, porcines, ovines, caprines, mammalian laboratory animals, mammalian farm animals, mammalian sport animals, and mammalian pets.

As used herein, the terms “administration” and “administering” refer to the act of giving a drug, prodrug, or other agent, or therapeutic treatment (e.g., SP-A peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., multiple SP-A peptides or an SP-A peptide and another therapeutic agent) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

“Treatment,” as used herein, covers any administration or application of a therapeutic for disease in a mammal, including a human, and includes inhibiting the disease, arresting its development, or relieving the disease, for example, by causing regression, or restoring or repairing a lost, missing, or defective function; or stimulating an inefficient process.

A “pharmaceutically acceptable carrier” refers to a non-toxic solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

### DETAILED DESCRIPTION OF THE INVENTION

As noted, experiments conducted during the course of developing embodiments for the present invention developed small molecules with improved stability and bio-availability, derived from the SP-A active site, as therapeutics targeting eosinophil normalization. Indeed, such experiments screened the 10-mer and 20-mer SP-A peptides, along with a series of peptidomimetics derived from full-length SP-A for their direct apoptosis-promoting functions on eosinophils. Several potential peptidomimetics were identified that closely resemble full-length SP-A in reference to the cytotoxic effect of SP-A on eosinophils in vitro. Such results represent a proof-of-concept that small molecules derived from the active site of SP-A possess activity against eosinophils and paves the way for development of a new class of therapeutics for allergic airway inflammation.

Accordingly, provided herein are compositions and methods for treating and preventing lung disease. In particular, provided herein are SP-A peptides and uses thereof in the treatment and prevention of lung disease (e.g., asthma).

In certain embodiments, the present invention provides a treatment for asthma using peptide analogues whose sequence is derived or adapted from the active region of endogenous human SP-A that contains the major Q allele at position 223 of the SP-A2 peptide. For example, in some embodiments, a composition comprising a peptide comprising, consisting essentially of, or consisting of an amino acid sequence selected from, for example, Ac-KEQCVEMYTD-NH₂ (SEQ ID NO: 2), Ac-WGKEQCVEMYTD- NH₂ (SEQ ID NO: 3), (Ac-KEQCVEMYTD-NH₂)₂ (SEQ ID NO: 4), Ac-KEQCVEMYTD-acid (SEQ ID NO: 5), H-KEQCVEMYTD-acid (SEQ ID NO: 6), Ac-KEQCVE-Nle-YTD-NH₂ (SEQ ID NO: 7), Ac-KEQSVEMYTD-NH₂ (SEQ ID NO: 8), Ac-KEQAVEMYTD-NH₂ (SEQ ID NO: 9), Ac-SDGTPVNYTNWYRGEPAGRGKEQ-NH₂ (SEQ ID NO: 10), Ac-GDFRYSDGTPVNYTNWYRGE-NH₂ (SEQ ID NO: 11), Ac-WGKEQAVE-Nle-YTD-NH₂ (SEQ ID NO : 12), Ac-WGKEQCVE-Nle-YTD-NH₂ (SEQ ID NO: 13), Ac-RGKEQCVE-Nle-YTD-NH₂ (SEQ ID NO : 14), Ac-wGKEQCVE-Nle-YTD-NH₂ (SEQ ID NO: 15), or peptide analogues with at least 90% (e.g., 91%, 92%, 93%, 94%, 95%, 96%, 97%, 98%, 99%, or 100%) identity to the peptides is provided. Further embodiments provide a composition consisting essentially of a peptide analogue selected from, for example, Ac-KEQCVEMYTD-NH₂ (SEQ ID NO: 2), Ac-WGKEQCVEMYTD- NH₂ (SEQ ID NO: 3), (Ac-KEQCVEMYTD-NH₂)₂ (SEQ ID NO: 4), Ac-KEQCVEMYTD-acid (SEQ ID NO: 5), H-KEQCVEMYTD-acid (SEQ ID NO: 6), Ac-KEQCVE-Nle-YTD-NH₂ (SEQ ID NO: 7), Ac-KEQSVEMYTD-NH₂ (SEQ ID NO: 8), Ac-KEQAVEMYTD-NH₂ (SEQ ID NO: 9), Ac-SDGTPVNYTNWYRGEPAGRGKEQ-NH₂ (SEQ ID NO: 10), Ac-GDFRYSDGTPVNYTNWYRGE-NH₂ (SEQ ID NO: 11), Ac-WGKEQAVE-Nle-YTD-NH₂ (SEQ ID NO : 12), Ac-WGKEQCVE-Nle-YTD-NH₂ (SEQ ID NO: 13), Ac-RGKEQCVE-Nle-YTD-NH₂ (SEQ ID NO : 14), Ac-wGKEQCVE-Nle-YTD-NH₂ (SEQ ID NO: 15). In some embodiments, the peptide binds to a receptor selected from, for example FC (CD16/32), Sirp-alpha, TLR-2, or EGFR.

The present invention further provides variants and mimetics of the SP-A peptides described herein. In some embodiments, an SP-A peptide comprises conservative, semi- conservative, and/or non-conservative substitutions relative to the peptides described herein (e.g., at positions involved in SP-A signaling or positions not involved in SP-A signaling).

Embodiments are not limited to specific substitutions. In some embodiments, the peptides described herein are further modified (e.g., substitution, deletion, or addition of standard amino acids; chemical modification; etc.). Modifications that are understood in the field include N-terminal modification, C-terminal modification (which protects the peptide from proteolytic degradation), alkylation of amide groups, hydrocarbon “stapling” (e.g., to stabilize alpha-helix conformations). In some embodiments, the peptides described herein may be modified by conservative residue substitutions, for example, of the charged residues (K to R, R to K, D to E and E to D). In some embodiments, such conservative substitutions provide subtle changes, for example, to the receptor binding sites with the goal of improving specificity and/or biological activity. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily-skilled peptide chemist. The a-carbon of an amino acid may be mono- or dimethylated.

In some embodiments, one or more intra-peptide disulfide bonds are introduced (e.g., between two cysteines within the peptide. In some embodiments, the presence of an intra- peptide disulfide bond stabilizes the peptide.

In some embodiments, any embodiments described herein may comprise peptidomimetics corresponding to the peptides described herein with various modifications that are understood in the field. In some embodiments, residues in the peptide sequences described herein may be substituted with amino acids having similar characteristics (e.g., hydrophobic to hydrophobic, neutral to neutral, etc.) or having other desired characteristics (e.g., more acidic, more hydrophobic, less bulky, more bulky, etc.). In some embodiments, non-natural amino acids (or naturally-occurring amino acids other than the standard 20 amino acids) are substituted in order to achieve desired properties.

In some embodiments, residues having a side chain that is positively charged under physiological conditions, or residues where a positively-charged side chain is desired, are substituted with a residue including, but not limited to: lysine, homolysine, δ-hydroxylysine, homoarginine, 2,4-diaminobutyric acid, 3-homoarginine, D-arginine, arginal (—COOH in arginine is replaced by —CHO), 2-amino-3-guanidinopropionic acid, nitroarginine (N(G)-nitroarginine), nitrosoarginine (N(G)-nitrosoarginine), methylarginine (N-methyl-arginine), ϵ-N-methyllysine, allo-hydroxylysine, 2,3-diaminopropionic acid, 2,2′-diaminopimelic acid, ornithine, sym-dimethylarginine, asym-dimethylarginine, 2,6-diaminohexinic acid, p-aminobenzoic acid and 3-aminotyrosine and, histidine, 1-methylhistidine, and 3-methylhistidine. A neutral residue is a residue having a side chain that is uncharged under physiological conditions. A polar residue preferably has at least one polar group in the side chain. In some embodiments, polar groups are selected from hydroxyl, sulfhydryl, amine, amide and ester groups or other groups which permit the formation of hydrogen bridges.

In some embodiments, residues having a side chain that is neutral/polar under physiological conditions, or residues where a neutral side chain is desired, are substituted with a residue including, but not limited to: asparagine, cysteine, glutamine, serine, threonine, tyrosine, citrulline, N-methylserine, homoserine, allo-threonine and 3,5-dinitro-tyrosine, and β-homoserine.

Residues having a non-polar, hydrophobic side chain are residues that are uncharged under physiological conditions, preferably with a hydropathy index above 0, particularly above 3. In some embodiments, non-polar, hydrophobic side chains are selected from alkyl, alkylene, alkoxy, alkenoxy, alkylsulfanyl and alkenylsulfanyl residues having from 1 to 10, preferably from 2 to 6, carbon atoms, or aryl residues having from 5 to 12 carbon atoms. In some embodiments, residues having a non-polar, hydrophobic side chain are, or residues where a non-polar, hydrophobic side chain is desired, are substituted with a residue including, but not limited to: leucine, isoleucine, valine, methionine, alanine, phenylalanine, N-methylleucine, tert-butylglycine, octylglycine, cyclohexylalanine, β-alanine, 1-aminocyclohexylcarboxylic acid, N-methylisoleucine, norleucine, norvaline, and N-methylvaline.

In some embodiments, peptide and polypeptides are isolated and/or purified (or substantially isolated and/or substantially purified). Accordingly, in such embodiments, peptides and/or polypeptides are provided in substantially isolated form. In some embodiments, peptides and/or polypeptides are isolated from other peptides and/or polypeptides as a result of solid phase peptide synthesis, for example. Alternatively, peptides and/or polypeptides can be substantially isolated from other proteins after cell lysis from recombinant production. Standard methods of protein purification (e.g., HPLC) can be employed to substantially purify peptides and/or polypeptides. In some embodiments, the present invention provides a preparation of peptides and/or polypeptides in a number of formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations may contain protective agents, such as buffers, preservatives, cryprotectants (e.g., sugars such as trehalose), etc. The form of such preparations can be solutions, gels, etc. In some embodiments, peptides and/or polypeptides are prepared in lyophilized form. Moreover, such preparations can include other desired agents, such as small molecules or other peptides, polypeptides or proteins. Indeed, such a preparation comprising a mixture of different embodiments of the peptides and/or polypeptides described here may be provided.

In some embodiments, provided herein are peptidomimetic versions of the peptide sequences described herein or variants thereof. In some embodiments, a peptidomimetic is characterized by an entity that retains the polarity (or non-polarity, hydrophobicity, etc.), three-dimensional size, and functionality (bioactivity) of its peptide equivalent but wherein all or a portion of the peptide bonds have been replaced (e.g., by more stable linkages). In some embodiments, ‘stable’ refers to being more resistant to chemical degradation or enzymatic degradation by hydrolytic enzymes. In some embodiments, the bond which replaces the amide bond (e.g., amide bond surrogate) conserves some properties of the amide bond (e.g., conformation, steric bulk, electrostatic character, capacity for hydrogen bonding, etc.). Chapter 14 of “Drug Design and Development”, Krogsgaard, Larsen, Liljefors and Madsen (Eds) 1996, Horwood Acad. Publishers provides a general discussion of techniques for the design and synthesis of peptidomimetics and is herein incorporated by reference in its entirety. Suitable amide bond surrogates include, but are not limited to: N-alkylation (Schmidt, R. et al., Int. J. Peptide Protein Res., 1995, 46,47; herein incorporated by reference in its entirety), retro-inverse amide (Chorev, M. and Goodman, M., Acc. Chem. Res, 1993, 26, 266; herein incorporated by reference in its entirety), thioamide (Sherman D. B. and Spatola, A. F. J. Am. Chem. Soc., 1990, 112, 433; herein incorporated by reference in its entirety), thioester, phosphonate, ketomethylene (Hoffman, R. V. and Kim, H. O. J. Org. Chem., 1995, 60, 5107; herein incorporated by reference in its entirety), hydroxymethylene, fluorovinyl (Allmendinger, T. et al., Tetrahydron Lett., 1990, 31, 7297; herein incorporated by reference in its entirety), vinyl, methyleneamino (Sasaki, Y and Abe, J. Chem. Pharm. Bull. 1997 45, 13; herein incorporated by reference in its entirety), methylenethio (Spatola, A. F., Methods Neurosci, 1993, 13, 19; herein incorporated by reference in its entirety), alkane (Lavielle, S. et. al., Int. J.Peptide Protein Res., 1993, 42, 270; herein incorporated by reference in its entirety) and sulfonamido (Luisi, G. et al. Tetrahedron Lett. 1993, 34, 2391; herein incorporated by reference in its entirety).

As well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Suitable peptidomimetics include reduced peptides where the amide bond has been reduced to a methylene amine by treatment with a reducing agent (e.g. borane or a hydride reagent such as lithium aluminum-hydride); such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142; herein incorporated by reference in its entirety.

Any carrier which can supply an active peptide or polypeptide (e.g., without destroying the peptide or polypeptide within the carrier) is a suitable carrier, and such carriers are well known in the art. In some embodiments, compositions are formulated for administration by any suitable route, including but not limited to, orally (e.g., such as in the form of tablets, capsules, granules or powders), sublingually, buccally, parenterally (such as by subcutaneous, intravenous, intramuscular, intradermal, or intrasternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), etc.

A pharmaceutical composition may be administered in the form, which is formulated with a pharmaceutically acceptable carrier and optional excipients, adjuvants, etc. in accordance with good pharmaceutical practice. The peptide-based pharmaceutical composition may be in the form of a solid, semi-solid or liquid dosage form: such as powder, solution, elixir, syrup, suspension, cream, drops, paste and spray. As those skilled in the art would recognize, depending on the chosen route of administration (e.g. pill, injection, etc.), the composition form is determined. In general, it is preferred to use a unit dosage form in order to achieve an easy and accurate administration of the active pharmaceutical peptide or polypeptide. In general, the therapeutically effective pharmaceutical compound is present in such a dosage form at a concentration level ranging from about 0.5% to about 99% by weight of the total composition, e.g., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, an peptides-based pharmaceutical composition is provided in a unit dosage form for administration to a subject, comprising a peptides or polypeptide and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions. The peptides and the polypeptides encompassing a substantially alpha helical peptide region that are disclosed herein may be further derivatized by chemical alterations, such as amidation, glycosylation, acylation, sulfation, phosphorylation, acetylation, and cyclization. Such chemical alterations can be imparted through chemical or biochemical methodologies, as well as through in vivo processes, or any combination thereof.

The peptides and polypeptides described herein may be prepared as salts with various inorganic and organic acids and bases. Such salts include salts prepared with organic and inorganic acids, for example, with HCl, HBr, H₂SO₄, H₃PO₄, trifluoroacetic acid, acetic acid, formic acid, methanesulfonic acid, toluenesulfonic acid, maleic acid, fumaric acid and camphorsulfonic acid. Salts prepared with bases include ammonium salts, alkali metal salts, e.g. sodium and potassium salts, alkali earth salts, e.g. calcium and magnesium salts, and zinc salts. The salts may be formed by conventional means, such as by reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

The peptides and polypeptides described herein can be formulated as pharmaceutically acceptable salts and/or complexes thereof. Pharmaceutically acceptable salts include acid addition salts such as those containing sulfate, hydrochloride, phosphate, sulfamate, acetate, citrate, lactate, tartrate, succinate, oxalate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

The peptides and polypeptides described herein may be formulated as pharmaceutical compositions for use in conjunction with the methods of the present disclosure. Compositions disclosed herein may conveniently be provided in the form of formulations suitable for parenteral administration, including subcutaneous, intramuscular and intravenous administration, nasal administration, pulmonary administration, or oral administration. Suitable formulation of peptides and polypeptides for each such route of administration is described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1988).

Certain of the peptides and polypeptides described herein may be substantially insoluble in water and sparingly soluble in most pharmaceutically acceptable protic solvents and in vegetable oils. In certain embodiments, cyclodextrins may be added as aqueous solubility enhancers. Cyclodextrins include methyl, dimethyl, hydroxypropyl, hydroxyethyl, glucosyl, maltosyl and maltotriosyl derivatives of alpha-, beta-, and gamma-cyclodextrin. An exemplary cyclodextrin solubility enhancer is hydroxypropyl-beta-cyclodextrin (HPBCD), which may be added to any of the above-described compositions to further improve the aqueous solubility characteristics of the peptides or polypeptides. In one embodiment, the composition comprises 0.1% to 20% HPBCD, 1% to 15% HPBCD, or from 2.5% to 10% HPBCD. The amount of solubility enhancer employed will depend on the amount of peptide or polypeptide of the present disclosure in the composition. In certain embodiments, the peptides may be formulated in non-aqueous polar aprotic solvents such as DMSO, dimethylformamide (DMF) or N-methylpyrrolidone (NMP).

In some cases, it will be convenient to provide the peptide or polypeptide and another active agent in a single composition or solution for administration together. In other cases, it may be more advantageous to administer the additional agent separately from said polypeptide. For use, pharmaceutical compositions of the peptides and polypeptides described herein may be provided in unit dosage form containing an amount of the peptide or polypeptide effective for a single administration. Unit dosage forms useful for subcutaneous administration include prefilled syringes and injectors.

In certain embodiments, the polypeptide is administered in an amount, expressed as a daily equivalent dose regardless of dosing frequency, of 50 micrograms (“mcg”) per day, 60 mcg per day, 70 mcg per day, 75 mcg per day, 100 mcg per day, 150 mcg per day, 200 mcg per day, or 250 mcg per day. In some embodiments, the polypeptide is administered in an amount of 500 mcg per day, 750 mcg per day, or 1 milligram (“mg”) per day. In yet further embodiments, the polypeptide is administered in an amount, expressed as a daily equivalent dose regardless of dosing frequency, of 1-10 mg per day, including 1 mg per day, 1.5 mg per day, 1.75 mg per day, 2 mg per day, 2.5 mg per day, 3 mg per day, 3.5 mg per day, 4 mg per day, 4.5 mg per day, 5 mg per day, 5.5 mg per day, 6 mg per day, 6.5 mg per day, 7 mg per day, 7.5 mg per day, 8 mg per day, 8.5 mg per day, 9 mg per day, 9.5 mg per day, or 10 mg per day. In various embodiments, the polypeptide is administered on a monthly dosage schedule. In other embodiments, the polypeptide is administered biweekly. In yet other embodiments, the polypeptide is administered weekly. In certain embodiments, the polypeptide is administered daily (“QD”). In select embodiments, the polypeptide is administered twice a day (“BID”). In typical embodiments, the polypeptide is administered for at least 3 months, at least 6 months, at least 12 months, or more. In some embodiments, the polypeptide is administered for at least 18 months, 2 years, 3 years, or more.

In one embodiment, the pharmaceutical compositions of this invention are suitable for inhaled administration. Suitable pharmaceutical compositions for inhaled administration will typically be in the form of an aerosol or a powder. Such compositions are generally administered using well-known delivery devices, such as a nebulizer inhaler, a metered-dose inhaler (MDI), a dry powder inhaler (DPI) or a similar delivery device.

In a specific embodiment of this invention, the pharmaceutical composition comprising the active agent is administered by inhalation using a nebulizer inhaler. Such nebulizer devices typically produce a stream of high velocity air that causes the pharmaceutical composition comprising the active agent to spray as a mist that is carried into the patient's respiratory tract. Accordingly, when formulated for use in a nebulizer inhaler, the active agent is typically dissolved in a suitable carrier to form a solution. Alternatively, the active agent can be micronized and combined with a suitable carrier to form a suspension of micronized particles of respirable size, where micronized is typically defined as having about 90% or more of the particles with a diameter of less than about 10 .mu.m. Suitable nebulizer devices are provided commercially, for example, by PART GmbH (Starnberg, German). Other nebulizer devices include Respimat (Boehringer Ingelheim) and those disclosed, for example, in U.S. Pat. No. 6,123,068 to Lloyd et al. and WO 97/12687 (Eicher et al.).

A representative pharmaceutical composition for use in a nebulizer inhaler comprises an isotonic aqueous solution comprising a SP-A peptide or a pharmaceutically acceptable salt or solvate or stereoisomer thereof.

In another specific embodiment of this invention, the pharmaceutical composition comprising the active agent is administered by inhalation using a dry powder inhaler. Such dry powder inhalers typically administer the active agent as a free-flowing powder that is dispersed in a patient's air-stream during inspiration. In order to achieve a free flowing powder, the active agent is typically formulated with a suitable excipient such as lactose or starch.

A representative pharmaceutical composition for use in a dry powder inhaler comprises dry lactose having a particle size between about 1 .mu.m and about 100 .mu.m and micronized particles of SP-A peptide, or a pharmaceutically acceptable salt or solvate or stereoisomer thereof.

Such a dry powder formulation can be made, for example, by combining the lactose with the active agent and then dry blending the components. Alternatively, if desired, the active agent can be formulated without an excipient. The pharmaceutical composition is then typically loaded into a dry powder dispenser, or into inhalation cartridges or capsules for use with a dry powder delivery device.

Examples of dry powder inhaler delivery devices include Diskhaler (GlaxoSmithKline, Research Triangle Park, N.C.) (see, e.g., U.S. Pat. No. 5,035,237 to Newell et al.); Diskus (GlaxoSmithKline) (see, e.g., U.S. Pat. No. 6,378,519 to Davies et al.); Turbuhaler (AstraZeneca, Wilmington, Del.) (see, e.g., U.S. Pat. No. 4,524,769 to Wetterlin); Rotahaler (GlaxoSmithKline) (see, e.g., U.S. Pat. No. 4,353,365 to Hallworth et al.) and Handihaler (Boehringer Ingelheim). Further examples of suitable DPI devices are described in U.S. Pat. No. 5,415,162 to Casper et al., U.S. Pat. No. 5,239,993 to Evans, and U.S. Pat. No. 5,715,810 to Armstrong et al., and references cited therein.

In yet another specific embodiment of this invention, the pharmaceutical composition comprising the active agent is administered by inhalation using a metered-dose inhaler. Such metered-dose inhalers typically discharge a measured amount of the active agent or a pharmaceutically acceptable salt or solvate or stereoisomer thereof using compressed propellant gas. Accordingly, pharmaceutical compositions administered using a metered-dose inhaler typically comprise a solution or suspension of the active agent in a liquefied propellant. Any suitable liquefied propellant may be employed including chlorofluorocarbons, such as CC1.sub.3F, and hydrofluoroalkanes (HFAs), such as 1,1,1,2-tetrafluoroethane (HFA 134a) and 1,1,1,2,3,3,3-heptafluoro-n-propane, (HFA 227). Due to concerns about chlorofluorocarbons affecting the ozone layer, formulations containing HFAs are generally preferred. Additional optional components of HFA formulations include co-solvents, such as ethanol or pentane, and surfactants, such as sorbitan trioleate, oleic acid, lecithin, and glycerin. See, for example, U.S. Pat. No. 5,225,183 to Purewal et al., EP 0717987 A2 (Minnesota Mining and Manufacturing Company), and WO 92/22286 (Minnesota Mining and Manufacturing Company).

A representative pharmaceutical composition for use in a metered-dose inhaler comprises from about 0.01% to about 5% by weight of a compound of SP-A peptide, or a pharmaceutically acceptable salt or solvate or stereoisomer thereof; from about 0% to about 20% by weight ethanol; and from about 0% to about 5% by weight surfactant; with the remainder being an HFA propellant.

Such compositions are typically prepared by adding chilled or pressurized hydrofluoroalkane to a suitable container containing the active agent, ethanol (if present) and the surfactant (if present). To prepare a suspension, the active agent is micronized and then combined with the propellant. The formulation is then loaded into an aerosol canister, which forms a portion of a metered-dose inhaler device. Examples of metered-dose inhaler devices developed specifically for use with HFA propellants are provided in U.S. Pat. No. 6,006,745 to Marecki and U.S. Pat. No. 6,143,277 to Ashurst et al. Alternatively, a suspension formulation can be prepared by spray drying a coating of surfactant on micronized particles of the active agent. See, for example, WO 99/53901 (Glaxo Group Ltd.) and WO 00/61108 (Glaxo Group Ltd.).

For additional examples of processes of preparing respirable particles, and formulations and devices suitable for inhalation dosing see U.S. Pat. No. 6,268,533 to Gao et al., U.S. Pat. No. 5,983,956 to Trofast, U.S. Pat. No. 5,874,063 to Briggner et al., and U.S. Pat. No. 6,221,398 to Jakupovic et al.; and WO 99/55319 (Glaxo Group Ltd.) and WO 00/30614 (AstraZeneca AB).

In some embodiments, peptides/polypeptides are provided in pharmaceutical compositions and/or co-administered (concurrently or in series) with one or more additional therapeutic agents. Such additional agents may be for treatment or prevention of lung inflammation (e.g., asthma). Additional agents may include, but are not limited to: Short-acting beta2-adrenoceptor agonists (SABA), such as salbutamol (albuterol USAN); anticholinergic medications, such as ipratropium bromide, inhaled epinephrine, inhaled or systemic corticosteroids; leukotriene receptor antagonists (e.g., montelukast and zafirlukast); and combinations thereof.

In some embodiments, provided herein are methods for treating patients suffering from (or at risk of) lung disease (e.g., asthma) and/or in need of treatment (or preventative therapy). In some embodiments, subjects are obese or are not obese. In some embodiments, subjects are identified as having an SP-A genotype associated with increased risk of asthma or severe asthma (e.g., those genotypes described herein).

In some embodiments, a pharmaceutical composition comprising at least one SP-A peptide or polypeptide described herein is delivered to such a patient in an amount and at a location sufficient to treat the condition. In some embodiments, peptides and/or polypeptides (or pharmaceutical composition comprising such) can be delivered to the patient systemically or locally, and it will be within the ordinary skill of the medical professional treating such patient to ascertain the most appropriate delivery route, time course, and dosage for treatment. It will be appreciated that application methods of treating a patient most preferably substantially alleviates or even eliminates such symptoms; however, as with many medical treatments, application of the inventive method is deemed successful if, during, following, or otherwise as a result of the inventive method, the symptoms of the disease or disorder in the patient subside to an ascertainable degree.

The present disclosure is not limited to the treatment of asthma. Any inflammatory conditions known in the art or otherwise contemplated herein may be treated in accordance with the presently disclosed and claimed inventive concept(s). Non-limiting examples of disease conditions having inflammation associated therewith include infection-related or non-infectious inflammatory conditions in the lung (e.g., asthma, sepsis, chronic obstructive pulmonary disease (COPD), lung infections, Respiratory Distress Syndrome, bronchopulmonary dysplasia, etc.); infection-related or non-infectious inflammatory conditions in other organs (e.g., colitis, Inflammatory Bowel Disease, diabetic nephropathy, hemorrhagic shock); inflammation-induced cancer (i.e., cancer progression in patients with colitis or Inflammatory Bowel Disease); and the like.

### EXPERIMENTAL

### Example I

This example decries the materials and methods utilized in Example II.

### Eosinophil Isolation

IL-5 transgenic mice were euthanized and blood collected by cardiac puncture through the left ventricle. Red blood cells (RBCs) were lysed using a red blood cell lysis solution (Miltenyi Biotec, Auburn CA). Eosinophils were isolated by negative selection using biotin-conjugated antibodies (CD45R, Thy 1.2, F4/80) and magnetic beads, as previously described (see, Dy, A. B. C., et al., 2019 J Immunol 203: 1122-1130; Ledford, J. G., et al., 2012 PLoS One 7: e32436). Purity of each preparation was verified using standard morphometric analysis of cytocentrifuged slides stained with Easy III™ rapid differential staining kit (Azer Scientific, Morgantown Pa.) to be greater than 95%.

### Generation of 10 and 20 Amino Acid Peptides Derived From Full-Length SP-A

Ten- and 20-mer amino acid peptides were custom-synthesized (Genscript Biotech Corporation, Piscataway N.J.) and verified to be 98.8% and 98.0% pure, respectively. Each vial of lyophilized 10-mer peptide was reconstituted using sterile-filtered PBS (Gibco, Gaithersburg Md.) to an initial concentration of 2 mg/ml, while each vial of lyophilized 20-mer peptide was reconstituted using molecular biology grade H₂O (Corning, Tewksbury Mass.) to an initial concentration of 2 mg/ml. The choice of solvent was based on solubility reports provided by Genscript.

### Generation of Peptidomimetics

Peptidomimetics were synthesized by solid phase methodology in the Ligand Discovery Laboratory (The University of Arizona, Tucson Ariz.). The peptidomimetics were designed to be small molecule derivatives that mimic the mature SP-A active site (KEQCVEMYTD) with improved stability and bioavailability. Products were purified by high-performance liquid chromatography (HPLC) and its structures analyzed by nuclear magnetic resonance (NMR) spectroscopy and liquid chromatography — mass spectrometry (LC-MS). Each vial of lyophilized peptidomimetic was reconstituted using molecular biology grade H₂O (Corning, Tewksbury Mass.) and a maximum final concentration of 10mM DMSO (Sigma, St. Louis Mo.) to an initial concentration of 1 mg/ml.

### Assessment of Eosinophil Cytotoxicity by Real-Time Impedance Tracing

High throughput real-time monitoring of eosinophil cell death was assessed by measuring electrical impedance using the xCELLigence Real-Time Cell Analyzer (ACEA Biosciences, San Diego Calif.) as previously described (see, Dy, A. B. C., et al., 2019 J Immunol 203: 1122-1130; Flynn, A. N., et al., FASEB J 27: 1498-1510; Zeng, C., et al., Environ Res 164: 452-458). Using 96-well gold electrode coated plates (E-plates, ACEA Biosciences) incubated at 37° C. and 5% CO₂, an initial background reading was obtained with media only. In a total volume of 100 eosinophils were seeded at 1×10⁶ cells/well and allowed to settle for ˜5 hours. Test compounds were added at various concentrations (1, 3, 10 and 30 μg/ml) and changes in electrical impedance were measured over time. Impedance measurements were calculated and presented as normalized cell index (see, Flynn, A. N., et al., FASEB J 27: 1498-1510; Zeng, C., et al., Environ Res 164: 452-458), where a decrease in cell index corresponds to an increase in eosinophil cytotoxicity. Impedance tracings over time were derived from averages of 3-4 technical replicates. To quantify and compare cytotoxicity, the area under the curve (AUC) was calculated using cell index values. Half maximal effective concentration (EC₅₀) values were generated using equivalent molar concentrations and their corresponding dose-response curves for each peptidomimetic.

### Statistical Analysis

All statistical analyses were performed using GraphPad Prism software. One-way ANOVA was used to assess global differences between samples, followed by multiple t-tests with Bonferroni's correction for multiple comparisons.

### Example II

Cytotoxic Effect of SP-A Derived Peptides Were Less in Magnitude Compared to Full-Length SP-A

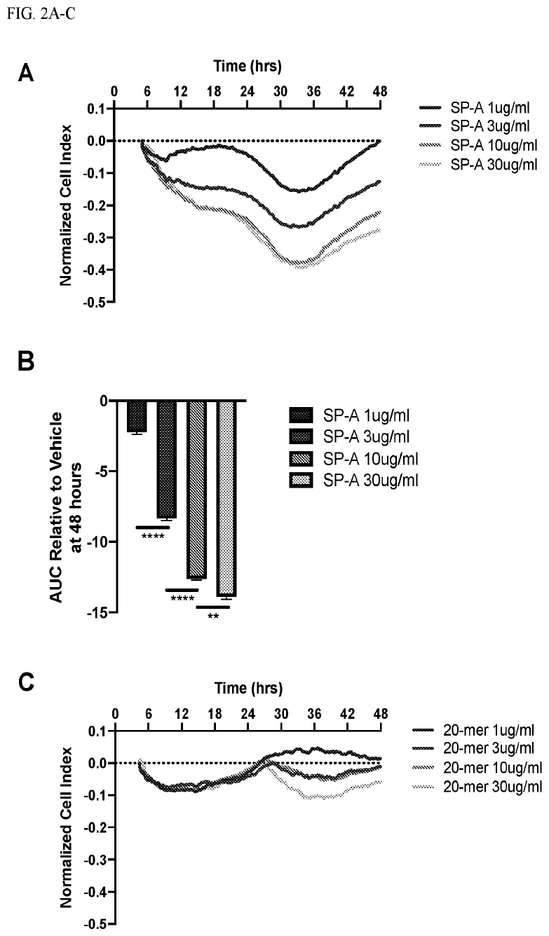

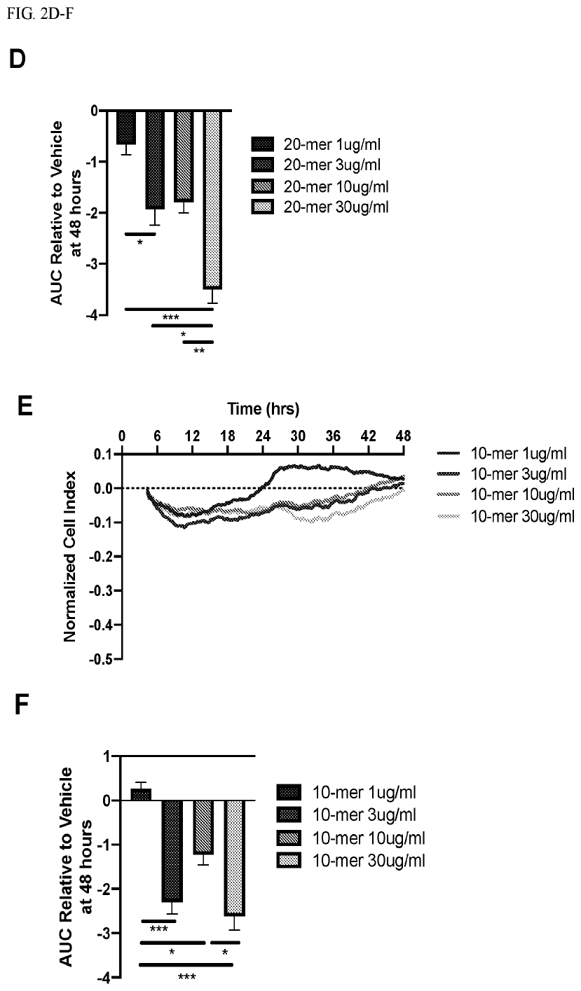

We first assessed the direct effect of these 10 and 20 amino acid peptides (10-mer and 20-mer) on eosinophil viability by RTCA. Similar to our previous results (see, Dy, A. B. C., et al., 2019 J Immunol 203: 1122-1130), the full-length SP-A induced eosinophil cell death in a dose-dependent manner, where addition of 30 μg/ml of SP-A resulted in a decrease in cell index corresponding to an average AUC of −13.89 (FIG. 2A, 2B). The addition of SP-A derived peptides, 10- and 20-mer, to eosinophils also resulted in increased cell death as indicated by negative AUC values. The average of the highest AUC magnitudes, corresponding to a concentration of 30 μg/ml of peptide, were -2.62 and -3.50, respectively (FIG. 2D, 2F). Normalized cell index tracings showed a decline in peptide activity after 24 hours, indicated by the increasing trend, for both the 10-mer and the 20-mer (FIG. 2C, 2E).

### Two Candidate Peptidomimetics Mimicked the Cytotoxic Effect of SPA at 3 μg/ml

To improve the stability of the SP-A derived peptides, peptidomimetics were synthesized for testing. There were 14 peptidomimetics that were initially screened. Peptidomimetic 856, 867, 868, 870 and 871 were modified from the original 10-mer native peptide residue by addition of an amine or acid group to the C-terminal, and acetylation or addition of a histidine to the N-terminal (Table 1). Peptidomimetic 882, 883, 884, 891, 892, 893 and 894 were modified from the original 10-mer native peptide residue by single amino acid substitutions (Table 1). Peptidomimetic 888 is a 23-amino acid sequence corresponding to position 181-203 of SP-A2, while peptidomimetic 889 is a 20-amino acid sequence corresponding to position 175-195 of SP-A2 (Table 1). The peptidomimetic sequences and corresponding molecular weights are summarized in Table 1.

TABLE 1
Peptidomimetic sequences and molar concentrations. The 10-mer, 20-mer and
candidate peptidomimetics were resuspended at a concentration of 1 mg/ml. Their
corresponding molar concentrations were calculated based on their respective
molecular weights, w = D-tryptophan.

| | | | | Molar |
|---|---|---|---|---|
| | | | Molecular | Concentration |
| Mimetic ID | SEQ ID | | Weight | (pM) equivalent |
| # | Number | Sequence | (g/mol) | to 1 mg/ml |
| 10-mer | 16 | KEQCVEMYTD | 1250 | 800.00 |
| peptide | | | | |
| 20-mer | 17 | PAGRGKEQCVEMYTDGQWND | 2290 | 436.68 |
| peptide | | | | |
| 856 | 2 | Ac-KEQCVEMYTD-NH₂ | 1285 | 778.21 |
| 867 | 3 | Ac-WGKEQCVEMYTD-NH₂ | 1529 | 654.02 |
| 868 | 4 | (Ac-KEQCVEMYTD-NH₂)₂ | 2569 | 389.26 |
| 870 | 5 | Ac-KEQCVEMYTD-acid | 1286 | 777.61 |
| 871 | 6 | H-KEQCVEMYTD-acid | 1244 | 803.86 |
| 882 | 7 | Ac-KEQCVE-Nle-YTD-NH₂ | 1285 | 789.00 |
| 883 | 8 | Ac-KEQSVEMYTD-NH₂ | 1270 | 788.00 |
| 884 | 9 | Ac-KEQAVEMYTD-NH₂ | 1254 | 797.70 |
| 888 | 10 | Ac-SDGTPVNYTNWYRGEPAGRGKEQ- | 2622 | 381.39 |
| | | NH₂ | | |
| 889 | 11 | Ac-GDFRYSDGTPVNYTNWYRGE-NH₂ | 2437 | 410.34 |
| 891 | 12 | Ac-WGKEQAVE-Nle-YTD-NH₂ | 1479 | 676.27 |
| 892 | 13 | Ac-WGKEQCVE-Nle-YTD-NH₂ | 1511 | 661.94 |
| 893 | 14 | Ac-RGKEQCVE-Nle-YTD-NH₂ | 1481 | 675.36 |
| 894 | 15 | Ac-wGKEQCVE-Nle-YTD-NH₂ | 1511 | 661.94 |

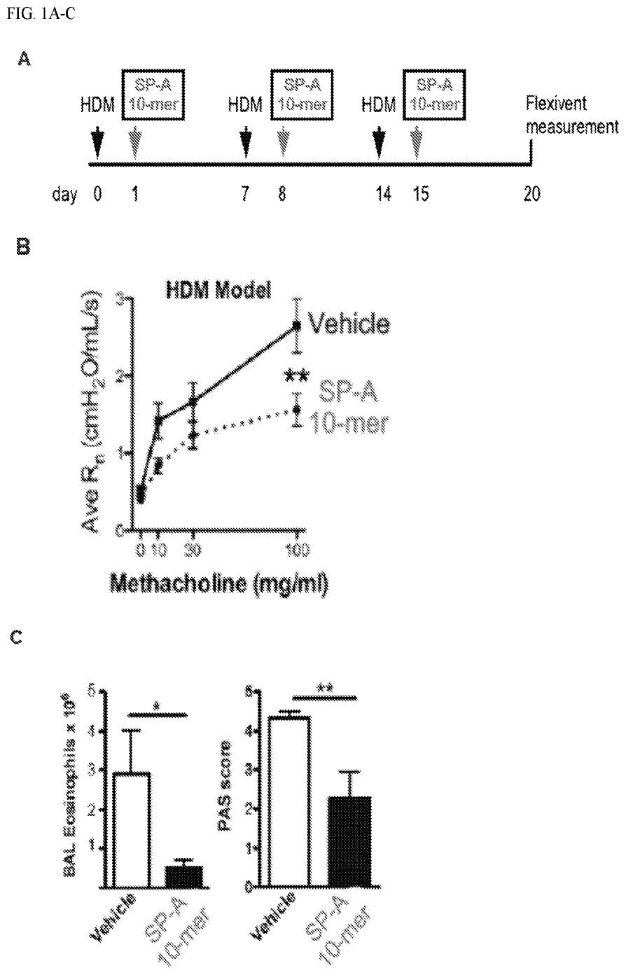

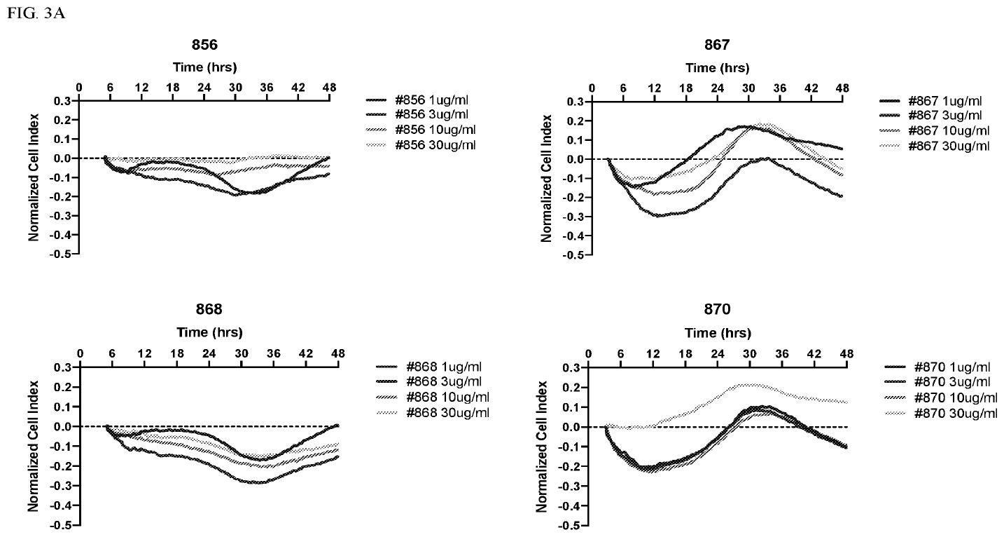

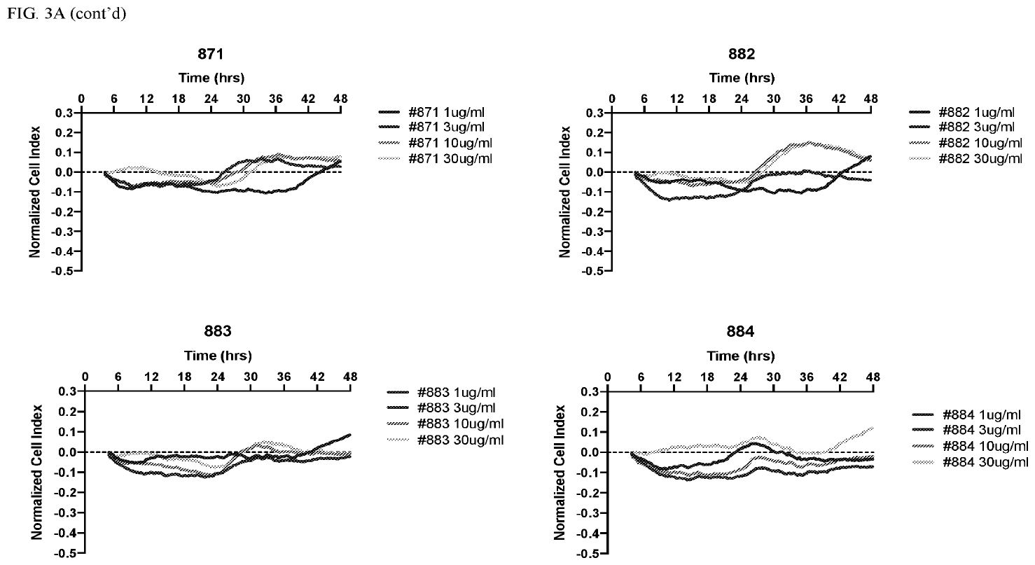

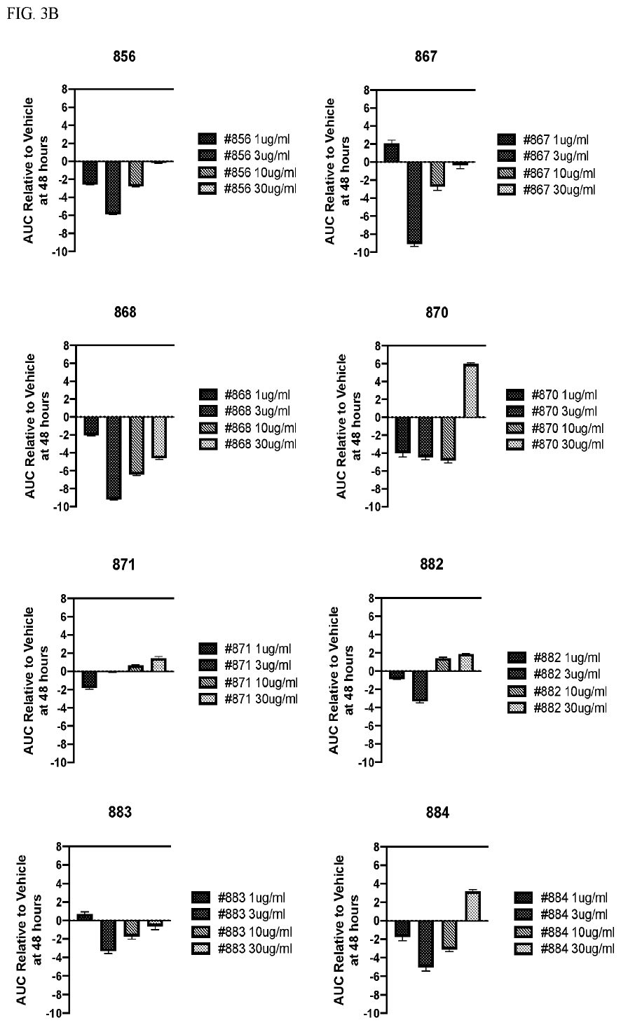

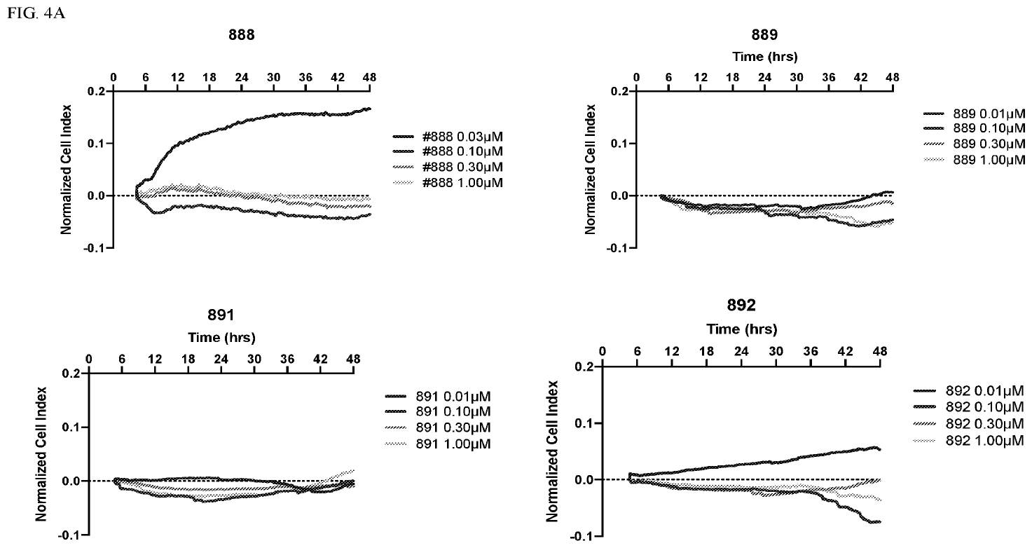

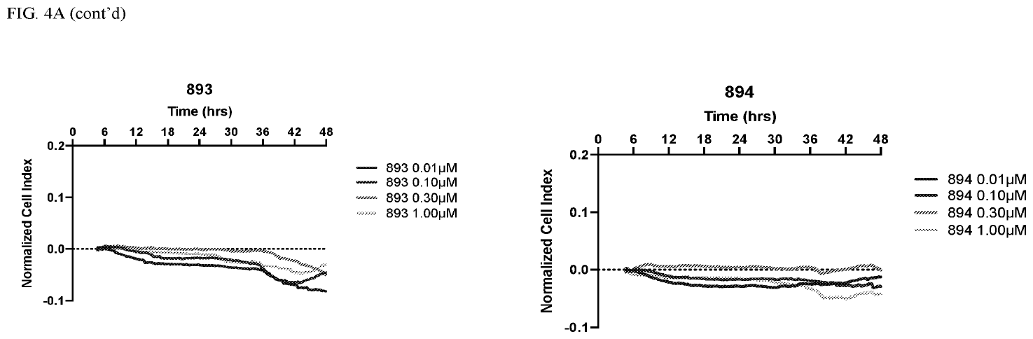

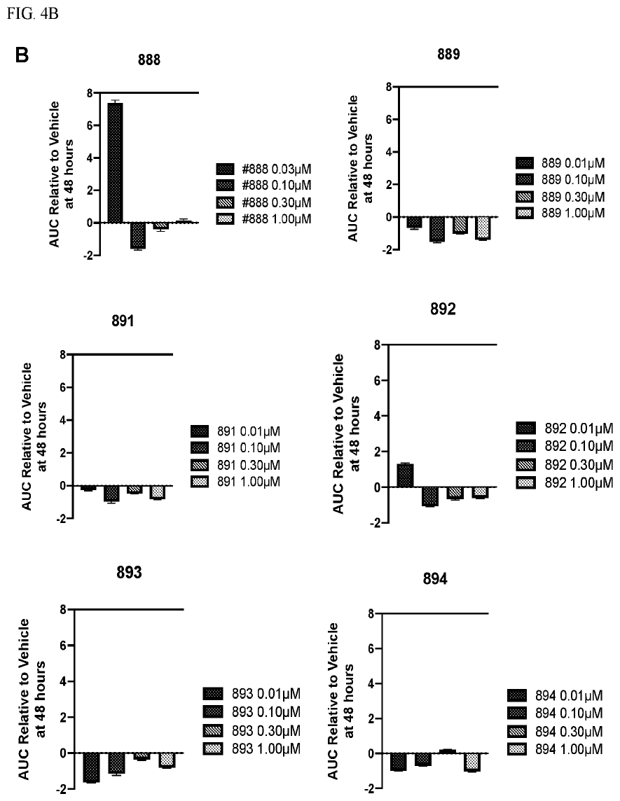

Using the same approach as FIG. 1, normalized cell indices and averages of the calculated AUCs are shown (FIGS. 3, 4). The mass concentration range used for the peptidomimetics in FIG. 3 is the range at which both the full-length SP-A and the 10-mer and 20-mer peptides were found to be active (see FIG. 2). However, to account for differences in the size between full-length SP-A and the various peptide sequence lengths, the equivalent molar concentration range at which full-length SP-A was found to be active were used in subsequent screening analyses (FIG. 4). Peptidomimetic 867 and 868 had the most robust cytotoxic effect on eosinophils at 3 μg/ml, as measured by AUC (FIGS. 3A, 2B)

### Calculated Half Maximal Effective Concentrations (EC₅₀) of Lead Peptidomimetics were Lower than Native 10- and 20-mer Peptides

Full-length SP-A is a much larger molecule compared to both the peptides and the peptidomimetics. Due to the discrepancies in size of the compounds tested, to be able to compare dose-response curves appropriately, molar concentrations for all compounds were calculated based on their respective molecular weights (Table 1). Full-length SP-A had an EC₅₀ of 0.158 μM (Table 2). Surprisingly, all of the peptidomimetics tested had EC₅₀ values lower than both the 10-mer and 20-mer peptides, with 892 and 894 having the two lowest values at 0.008 and 0.012 μM, respectively (Table 2).

TABLE 2
Half maximal effective concentrations (EC₅₀) based on dose-response
curves. Values for EC₅₀ were calculated using RTCA software
and were based on area under the curve (AUC) at each dose.
Concentration range used for full-length SP-A, 10-mer, 20-mer,
856, 867, 868, 870, 871, 882, 883 and 884 was 1, 3, 10 and
30 μg/ml. Concentration range used for 888, 889, 891, 892,
893 and 894 was 0.01, 0.10, 0.30 and 1.00 μM.

| Apoptosis inducer | EC₅₀ (μM) |
|---|---|
| Full-length SP-A | 0.158 |
| 20-mer peptide | 14.71 |
| 10-mer peptide | 16.01 |
| 856 | 8.207 |
| 867 | 0.950 |
| 868 | 10.92 |
| 870 | 10.75 |
| 871 | 2.518 |
| 882 | 3.000 |
| 883 | 9.412 |
| 884 | 10.63 |
| 888 | 0.035 |
| 889 | 1.788 |
| 891 | 1.026 |
| 892 | 0.008 |
| 893 | 0.038 |
| 894 | 0.012 |

### Discussion

It has recently been demonstrated that SP-A has the ability to promote eosinophil apoptosis and that this mechanism contributed to the clearance of eosinophils in the lung lumen after experimental allergy challenge (see, Dy, A. B. C., et al., 2019 J Immunol 203: 1122-1130). It was also demonstrated that this activity of SP-A was altered by genetic variation, where a glutamine is substituted for a lysine at position 223 of SP-A2. This suggests that the active site within SP-A that promotes apoptosis on eosinophils lies within this region, which motivated further investigation. First, peptides (10-mer and 20-mer) and peptidomimetics of this SP-A region were synthesized with the goal of improving stability while maintaining bioactivity. Second, these synthetic small molecules were tested for their ability to promote eosinophil cell death similarly to the full-length SP-A.

Experiments conducted herein demonstrate that a number of the synthesized small molecules were able to induce eosinophil cell death. Full-length SP-A served as the positive control, where an expected dose-dependent decrease in eosinophil viability as measured by RTCA was observed. Furthermore, tracings of the normalized cell index show an overall declining trend, suggesting a consistent and continuous death-inducing effect over the course of 48 hours. The 10- and 20-mer peptides derived from SP-A were likewise able to induce eosinophil cell death. However, comparing full-length SP-A and these peptides at each concentration, the degree of cell death induced by the peptides, indicated by the magnitude of the calculated AUCs, were less than that of the full-length SP-A. The AUC values of the peptides at 30 μg/ml were more comparable to the AUC at which 3 μg/ml of full-length SP-A was added. Additionally, the tracings of the normalized cell indices of the 10-mer and 20-mer suggest a less robust peptide activity compared to full-length SP-A, which may be an indicator of limited function in in vivo conditions.

Given these results, experiments were conducted that next opted to synthesize peptidomimetics to address this potential issue. Of the 14 peptidomimetics screened thus far, several peptidomimetics yielded promising results that will be evaluated further. Based on the magnitude of the AUCs at each dose, 867 and 868 had the most comparable response to full-length SP-A, whose AUC at 3 μg/ml is comparable to the AUC of the full-length SP-A at 30 μg/ml (see Table 3). Due to the large differences in molecular size between full-length SP-A and the synthesized molecules, calculations for half maximal effective concentrations (EC₅₀), which are important indicators of potency, were based on equivalent molar concentrations. Peptidomimetic 892 and 894 had the two lowest EC₅₀ of the 14 candidate molecules at 0.008 μM and 0.012 μM, respectively (see Table 3). However, despite the low EC₅₀, the magnitude of the calculated AUCs for both of these peptidomimetics are much smaller compared to full-length SP-A, 10-mer and 20-mer peptides and several peptidomimetics (see Table 3). This would suggest that, although peptidomimetic 892 and 894 require low concentrations to exert some degree of cytotoxicity on eosinophils, the effect is not as robust.

Taken together, because a goal was to identify candidate peptidomimetics that would recapitulate the cytotoxic activity of full-length SP-A on eosinophils, not only was it critical to derive EC₅₀ values as a measure of potency, but it is likewise essential to compare the magnitude of the changes in cell index as an indicator of effectivity. Therefore, four lead peptidomimetics (867, 868, 892 and 894 in Table 3) were identified.

In summary, experiments conducted herein provide evidence that small molecules derived from the active region in SP-A participating in its pro-apoptotic activity can induce similar effects on eosinophils. Future pre-clinical studies include further optimizing candidate peptidomimetics, validation in in vitro experiments using human eosinophils by flow cytometry and in vivo rescue experiments using animal models of asthma.

TABLE 3
Summary of characteristics of lead peptidomimetics. Average magnitude
of the largest AUC and their corresponding concentrations, along with
the EC₅₀ of each lead peptidomimetic are reported. Full-length
SP-A, 10-mer and 20-mer are also shown for comparison.

| | | Concentration at | |
|---|---|---|---|
| | | which average | |
| | Average magnitude | AUC is largest | |
| Compound | of largest AUC | (μM) | EC₅₀ |
| Full-length SP-A | −13.89 | 0.86 | 0.158 |
| 20-mer | −3.50 | 13.10 | 14.71 |
| 10-mer | −2.62 | 24.00 | 16.01 |
| 867 | −9.06 | 1.96 | 0.950 |
| 868 | −9.21 | 1.17 | 10.92 |
| 892 | −1.07 | 0.10 | 0.008 |
| 894 | −1.63 | 0.01 | 0.012 |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising a surfactant protein A (SP-A) peptide analogue comprising an amino acid sequence selected from the group consisting of Ac-KEQCVEMYTD-NH₂ (SEQ ID NO: 2), Ac-WGKEQCVEMYTD- NH₂ (SEQ ID NO: 3), (Ac-KEQCVEMYTD-NH₂)₂ (SEQ ID NO: 4), Ac-KEQCVEMYTD-acid (SEQ ID NO: 5), H-KEQCVEMYTD-acid (SEQ ID NO: 6), Ac-KEQCVE-Nle-YTD-NH₂ (SEQ ID NO: 7), Ac-KEQSVEMYTD-NH₂ (SEQ ID NO: 8), Ac-KEQAVEMYTD-NH₂(SEQ ID NO: 9), Ac-SDGTPVNYTNWYRGEPAGRGKEQ-NH₂ (SEQ ID NO: 10), Ac-GDFRYSDGTPVNYTNWYRGE-NH₂ (SEQ ID NO: 11), Ac-WGKEQAVE-Nle-YTD-NH₂ (SEQ ID NO : 12), Ac-WGKEQCVE-Nle-YTD-NH₂ (SEQ ID NO: 13), Ac-RGKEQCVE-Nle-YTD-NH₂ (SEQ ID NO : 14), Ac-wGKEQCVE-Nle-YTD-NH₂ (SEQ ID NO: 15), and peptides with at least 90% identity to said peptides.

2. The composition of claim 1, wherein said peptides are at least 95% identical to said peptides.

3. The composition of claim 1, wherein said peptides are 100% identical to said peptides.

4-7. (cancelled)

8. A system, comprising:
a) the composition of claim 1; and
b) a device for pulmonary delivery of said composition.

9. The system of claim 8, wherein said device is a metered dose inhaler.

10. A method of enhancing SP-A activity in a cell, comprising:
delivering the composition of claim 1 to said cell.

11. The method of claim 10, wherein said cell is a lung cell.

12. The method of claim 10, wherein said cell is in vivo.

13. The method of claim 9, wherein said composition reduces mucin production and/or reduces eosinophilia in said cell.

14. The method of claim 10, wherein said cell is in a subject diagnosed with asthma or COPD.

15. The method of claim 14, wherein said administering decreases or prevents symptoms or markers of asthma or COPD in said subject.

16. The method of claim 10, wherein said subject is obese.

17. The method of claim 10, wherein said peptide binds to a receptor selected from the group consisting of FC (CD16/32), Sirp-alpha, TLR-2 and EGFR.

18-33. (cancelled)
